(19) United States
(12) Patent Application Publication
(10) Pub. No.: US 2010/0211060 A1
(43) Pub. Date: Aug. 19, 2010

(54) RADIO FREQUENCY TREATMENT OF SUBCUTANEOUS FAT

(75) Inventors: Gareth Baron, Redwood City, CA (US); Walfre Franco, Foster City, CA (US); Jerzy Orkiszewski, Palo Alto, CA (US)

Correspondence Address: Cutera c/o MoFo, 425 Market Street, San Francisco, CA, 94105-2482, US

(73) Assignee: CUTERA, INC., Brisbane, CA (US)

(21) Appl. No.: 12/705,429

(22) Filed: Feb. 12, 2010

Related U.S. Application Data
(60) Provisional application No. 61/152,529, filed on Feb. 13, 2009.
(60) Provisional application No. 61/253,396, filed on Oct. 20, 2009.

(51) Int. Cl. A61B 18/18

(52) U.S. Cl. 606/33

(57) ABSTRACT

A method for treating subcutaneous fat with RF energy is disclosed. The method includes determining a thickness of a layer of fat of a portion of tissue to be treated. Based on the determined thickness, an RF frequency is selected. In particular, lower frequencies are used for thicker layers of fat while higher frequencies are used for thinner layers of fat. RF heating is applied to the portion of the tissue by applying RF energy at the selected RF frequency.

### CROSS REFERENCE TO RELATED APPLICATIONS

This application claims the benefit of prior copending U.S. Provisional Application Ser. No. 61/152,529, filed Feb. 13, 2009, and prior copending U.S. Provisional Application Ser. No. 61/253,396, filed Oct. 20, 2009, each of which is incorporated herein by reference in its entirety.

### BACKGROUND

Over the years, attempts have been made to use radiofrequency energy to reduce subcutaneous fat or cellulite. See for example, US. Patent Publication 2007/0282318, the disclosure of which is incorporated by reference.

In general, a handpiece is provided having an electrode system which is powered by an RF driver. If the electrode is monopolar, a return electrode, in the form of a large pad, is typically affixed to the patient's body remote from the treatment site. The large pad spreads out the energy to minimize heating. In contrast, the electrode in the handpiece has a much smaller surface area and therefore concentrates the RF energy in a smaller region. The RF energy coupled into the body heats the fat causing the cells to break down. Some prior art implementations use bipolar handpieces which do not require a separate return electrode.

In an effort to develop a system which can effectively reduce fat using an RF treatment protocol, the assignee herein has developed a number of various electrode designs and treatment modalities. For example, U.S. Patent Publication 2008/0312651 describes an RF electrode structure formed from concentric rings. U.S. Patent Publication 2009/0171346 describes a spiral RF electrode structure. Other features are described in the following pending U.S. Patent Publications 2009/0171341 and 2009/0171344; and pending U.S. patent application Ser. Nos. 12/144,948, filed Jun. 24, 2008; 12/134,119, filed Jun. 5, 2008 and 12/330,032, filed Dec. 8, 2008. All of these publications and applications are incorporated herein by reference in their entirety.

It is believed that most prior art systems which use RF energy in an attempt to reduce fat cells operate the RF electrode at a fixed, predetermined drive frequency. It is believed that an improved system would include the ability to deliver RF energy at different selected frequencies selected to best match the thickness of the fat layer being treated.

### SUMMARY OF THE INVENTION

In accordance with the subject invention, a RF treatment system is disclosed wherein the RF energy drive source is capable of generating a range of frequencies. In a preferred embodiment, the treatment frequency is selected based on the thickness of the fat layer to be treated. In some embodiments, the treatment frequency is selected based on a body measurement related to body weight. For example, for thicker layers of fat, lower frequencies would be used since lower frequencies tend to heat more uniformly both over the width of the electrode and down into the tissue. In contrast, the heating effect at higher frequencies tends to be more centrally located with respect to the electrode and maximized closer to the electrode surface. Such higher frequencies are therefore better suited for treating thinner layers of fat.

In a basic approach, the physician would determine the thickness of the fat layer by eye or with a tool, such as a calipers. The physician would then select a treatment frequency which best matched the fat layer thickness. In a more sophisticated approach, the handpiece itself can be provided with a means for measuring the thickness of the underlying fat layer and upon measurement, the system would automatically adjust the treatment frequency.

Further objects and advantages of the will become apparent based on the following detailed description, taken in conjunction with the drawings, in which:

### BRIEF DESCRIPTION OF THE DRAWINGS

FIG. 1 is a block diagram of a treatment system for implementing the subject invention.

FIG. 2 is perspective view of a handpiece where the outer shell is in phantom line.

FIG. 3 is a bottom view of the handpiece.

FIG. 4 is an exemplary geometry skin, fat and muscle.

FIG. 5 is a graph of normalized center-to-edge electric potential distributions across an RF electrode.

FIG. 6 includes graphs illustrating an internal electric field and power absorption within the subcutaneous fat layer.

FIG. 7 includes graphs illustrating temperature changes in ex vivo porcine tissue.

FIG. 8 includes radiometric temperature maps after RF exposure.

FIG. 9 includes illustrations showing resistive heating in subcutaneous tissue of a function of different RF frequencies.

FIG. 10 includes graphs showing resistive heating of subcutaneous tissue as a function of different RF frequencies.

FIG. 11 includes photographs of tissue treated at different RF frequencies.

FIG. 12 is a graph illustrating rations of power absorption as a function of frequency.

### DETAILED DESCRIPTION OF THE PREFERRED EMBODIMENTS

Radiofrequency (RF) heating is generated in tissue by energy transferred from the electric field to the tissue. Heat is generated by (i) the friction associated with the movement of the atoms and molecules in response to an electric field varying in time; and, (ii) collisions of the conduction charges with immobile atoms and molecules of the tissue structure.

RF technology offers unique advantages for non-invasive selective heating of relatively large volumes of subcutaneous adipose tissue. A properly induced electric field results in greater heating of fat than heating of skin and muscle. At low operational frequencies, the electric field is dominant in coupling energy into tissue. It then follows that relatively large volumes of fat can be heated with a monopolar device. Bulk tissue heating can be even further altered by a combination of coupling energy into tissue across the entire surface of an RF electrode while simultaneously controlling the surface distribution of the applied energy by adjusting the operational frequency.

Controlling the energy distribution on the skin surface provides a treatment for which the subcutaneous fat is selectively heated and the size of the heated volume is controllable. This treatment may allow for adjustment of the operational frequency to match anatomical structures from patient to patient or even within the same patient. For example, body areas with relative thin thickness of the subcutaneous fat layer would be treated using high frequencies while low frequencies would be used with thicker fat layers.

Another advantage of embodiments of the invention is that the depth of heating is electrically controlled. This gives the device the potential for greater ease of control, allowing it to be faster and more compact in size when compared to devices controlled by mechanical, rather than electrical means. An example of a device controlled by mechanical means is focus ultrasound devices, which are able to target different tissue depths by mechanically adjusting hand-piece components that change the depth of the focal point.

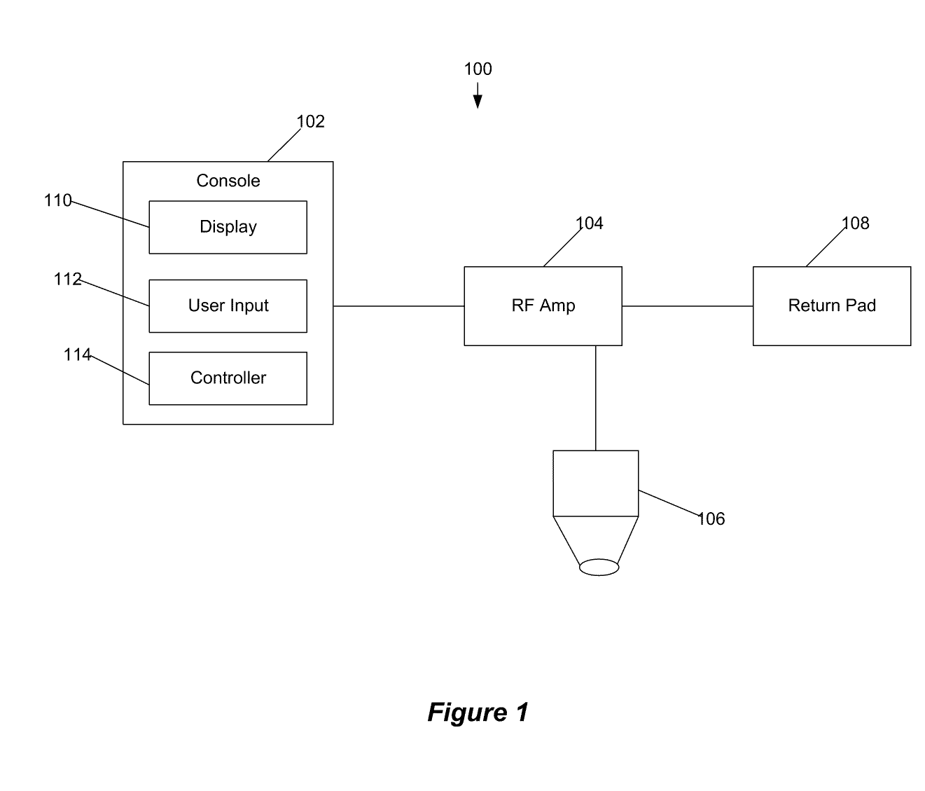

On the other hand, a system according to embodiments of the invention controlled by electrical means is illustrated in FIG. 1. FIG. 1 shows a simplified diagram of a system 100 for implementing the subject invention. The system includes a console 102 supplying control signals to an RF amp 104. Typically, the RF amp 104 would be located within console. The output from amp 104 is supplied to a handpiece 106 and a return pad 108.

Console 102 would include a display 110 and a user input region 112. In some cases, the user input can be via the touch screen display. A controller 114, in the form of a computer processor, controls the entire system. In addition to controlling the signals to the RF amp, the controller would communicate with the handpiece for controlling cooling and for safety monitoring as is well known in the art.

In accordance with the subject invention, the controller and RF amp are configured to be able to selectively generate a range of RF outputs. The controller can be programmed to digitally generate an RF signal of the desired frequency. This signal is supplied to the amp 104 where it is amplified and sent to the handpiece. In a preferred embodiment, the range of frequencies can be from 100 kilohertz to 10 megahertz but preferably should cover at least 500 kilohertz to 5 megahertz. In other embodiments, the range of frequencies can be from 500 kilohertz to 2 megahertz.

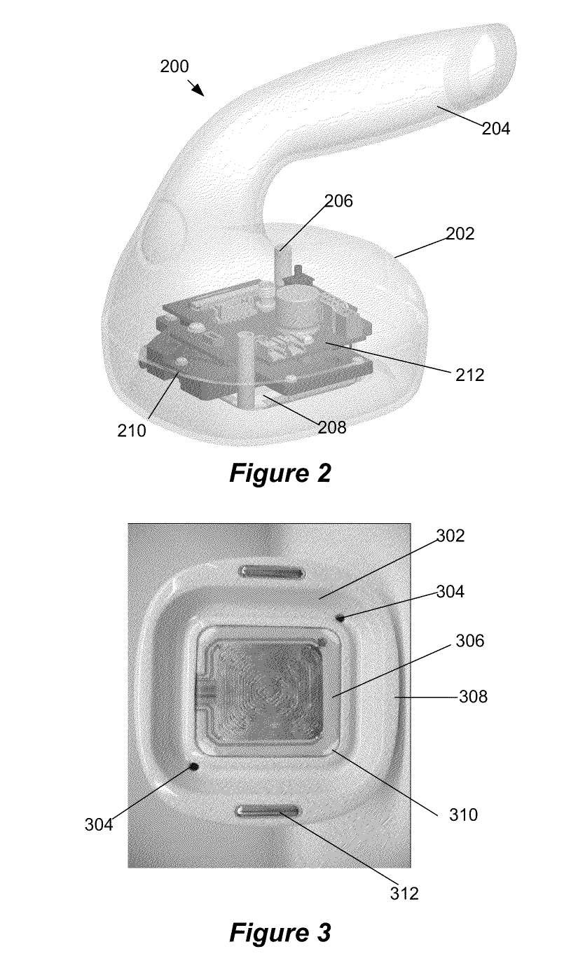

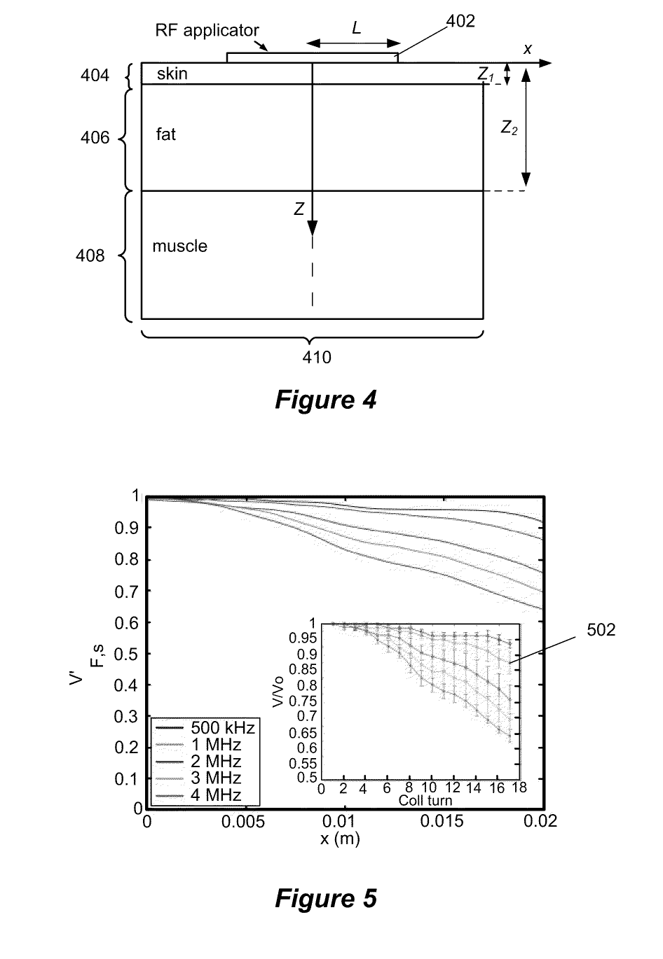

The subject invention will be useful in a wide variety of handpiece and electrode designs. It is believed that this approach can be used with both focused and unfocused systems. FIGS. 2 and 3 show a handpiece of the type which can be used with the subject system.

The handpiece 200 has a base section 202 and a handle 204. As best seen in FIG. 3, the bottom of the base includes a planar recessed section 302 surrounded by a raised rim 308. An RF electrode 306 is located in the center of the recess section 302. The electrode is monopolar and formed from copper. In embodiments of the invention, the geometry of the RF electrode 306 is a series of tightly spaced concentric rings designed to directly couple energy into tissue across the entire surface of the electrode in the handpiece 200. In this way, the design of the RF electrode 306 is capable of providing uniform heating, as opposed to edge heating that may be provided by ring-shaped and solid type RF electrodes. Uniform heating is achieved by controlling the distribution of the surface electric potential. Adjusting this distribution results in varying the extent of uniform heating and consequently, the size of the heating tissue volume. Further, operation at different selective frequencies based on characteristics of the portion of tissue to be treated affects the distribution of the applied surface electric potential.

A pair of vacuum ports 304 is located within the recessed region outside of the border of the electrode. The ports 304 are fluidly connected to channels 206 shown in FIG. 2. During use, suction is applied through the channels to draw the skin into close contact with the electrode. A transducer in the suction path can be used to measure the vacuum level which in turn provides a measurement of the level of contact between the patient's skin and the handpiece 200.

A plurality of thermistors 310 are provided for monitoring the temperature of the skin. If the skin becomes too hot, the system will shut down. Rollers 312 are provided on the bottom surface of the handpiece to facilitate movement of the handpiece over the tissue.

As seen in FIG. 2, an alumina heat spreader (not shown) is mounted to the top (inner) surface of the electrode. A thermoelectric cooler (TEC) 208 is mounted between the spreader and a heat sink 210. A circuit board 212 is mounted over the heat sink 210.

In use, the TEC cooler is first activated and suction is applied to cool the skin surface. When the desired surface temperature is reached, the RF energy is applied to the tissue. The RF energy couples into the tissues and provides heating. The cooled electrode produces a temperature gradient with the skin being much cooler than the underlying tissue.

Based on experiments, it has been determined that the temperature of the fat cells should be raised to at least about 50 degrees centigrade. Since fat cells have a higher impedance than other tissue, some selective heating is possible. Selective heating of fat is further aided by the fact there is little blood flow through fatty tissue. Blood flow can reduce tissue temperature.

During heating, the RF energy is supplied in cycles. For example, the RF energy is turned on for 1 to 10 seconds and then turned off for a similar time period. The total treatment time for each region is about one to three minutes. The amount of energy delivered depends on the power and frequency of the RF drive current. In general, it is expected that the handpiece will deliver about 10,000 joules over 16 cm² for a total fluence of about 600 joules/cm².

Of interest is that histological samples taken soon after treatment show little or no damage to the cells. However, samples taken many days after treatment (e.g. 9 to 14 days after), show significant cellular damage. It is believed that the RF treatment cause some initial damage to the walls of the fat cell nuclei which leads to a slower death of the cells.

In accordance with the subject invention, the RF frequency which is selected by the physician is tailored to the thickness of the fat layer. As discussed in more detail below, a lower frequency is selected for a thicker fat layer while a higher frequency is used for a thinner fat layer.

To determine the thickness of the layer of subcutaneous fat, an effective amount of RF energy along with a desired RF frequency, mathematically modeling may be utilized. From mathematical modeling and experimental data, it can be seen that as the frequency increases: (1) the electric potential at the skin surface decreases from the center to the edge of the RF electrode; (2) the difference between the power absorbed at the top and the bottom of the subcutaneous fat layer increases; (3) the difference between the power absorbed at the center and the periphery of the exposed subcutaneous fat volume also increases; and (4) the size of the heated subcutaneous fat volume decreases. As such, based on the portion of tissue where the RF heating is to be applied, differential delivery of heat is desired.

In biological tissue the rate of energy dissipated per unit volume at a given point is directly proportional to the electric conductivity of tissue and to the square of the induced internal electric field:

$${Q}_{F , i} ( x , z ) = \frac{1}{2} {σ}_{F , i} {{E}_{F , i} ( x , z )}^{2} , \qquad ( 1 )$$

where Q is the power absorption in watts per cubic meter (W/m3), σ is the electric conductivity in Siemens per meter (S/m), E is the internal electric field in volts per meter (V/m), the subindex F denotes the frequency in MHz and the subindex i represents skin, fat or muscle [s, f, m]. RF-induced internal electric fields can be modeled using the Laplace equation:

∇·(σ_{F,i}∇V_{F}(x, z))=0, (2)

where V is the electric potential in volts (V).

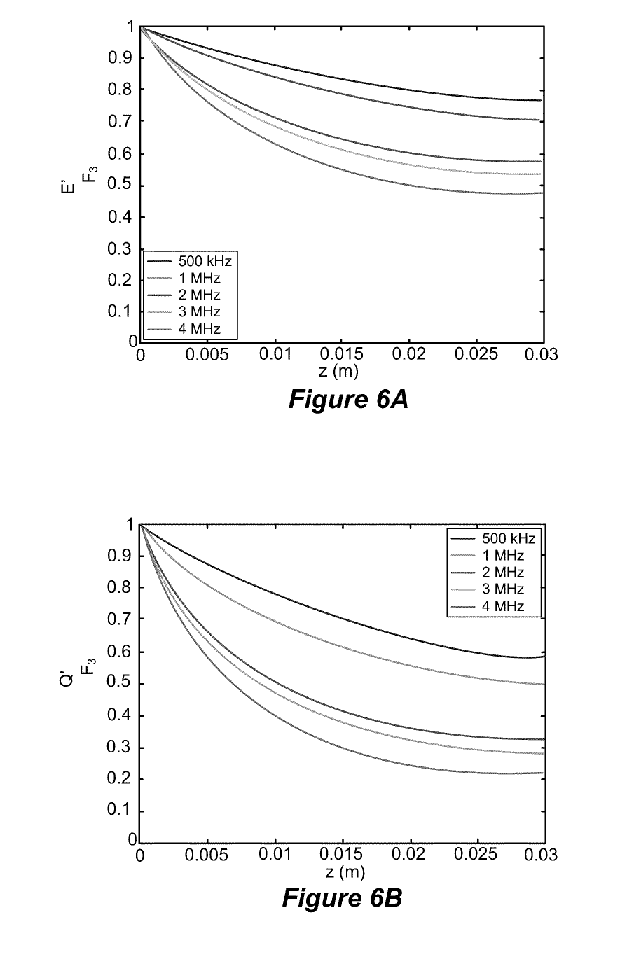

FIG. 4 illustrates the model geometry of biological tissue. The thickness of the skin 404 is 0.2 cm, the thickness of the fat 406 is 3 cm, and the thickness of the muscle 408 is 6.8 cm. The portion of tissue illustrated has a width 410 of 30 cm. RF electrode 402 is shown contacting the skin.

Changes in the surface distribution of the electric potential as a function of frequency can be modeled by modifying the surface boundary condition:

V_{F,s}(x,0)=V′_{F,s}(x), (3)

where V′ denotes experimental measurements obtained as described above. Mathematical model and modeling parameters are described in Franco W, Kothare A, Ronan S J, Grekin, R C and McCalmont T H. Selective heating of subcutaneous fat using a novel radiofrequency device: in vitro, in vivo and numerical feasibility studies. Lasers Surg Med, 2009. In Review, which is incorporated herein by reference.

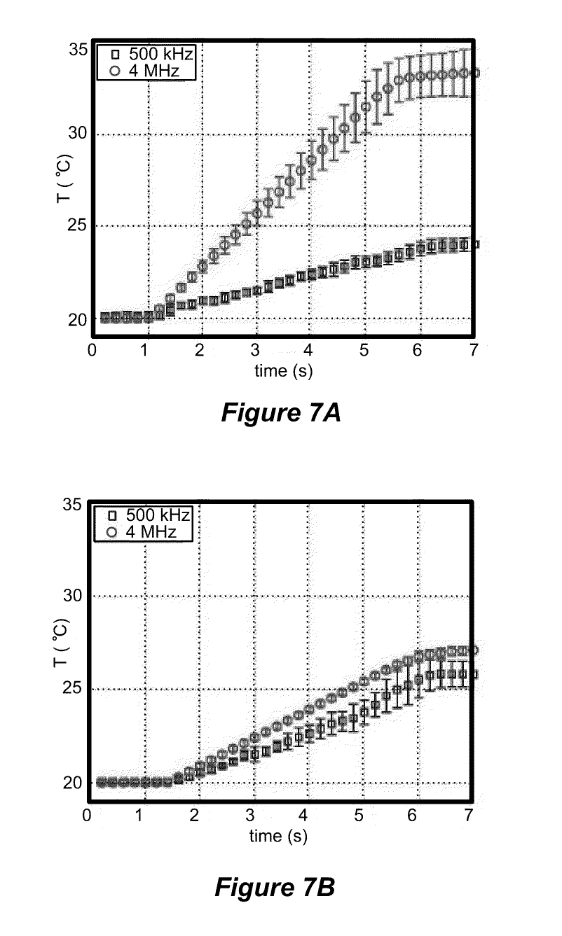

To experimentally show that frequency is related to body impedance, and therefore related to body anatomy and tissue structure, the electric potential between the RF electrode and the return path was measured from the center to the edge of the RF electrode at different frequencies: 500 kHz, 1, 2, 3, and 4 MHz. Electric potentials were measured at different powers. FIG. 5 illustrates these measurements. FIG. 5 shows a graph of normalized center-to-edge, along the x-axis with reference to FIG. 4, electric potential distributions, with respect to the center voltage V_{F},s(x, 0), across the RF electrode obtained from experimental measurements on human subjects at five operational frequencies: 500 kHz, 1, 2, 3 and 4 MHz. Insert 502 shows the corresponding standard deviations of the normalized curves average from four subjects. Qualitatively, it is illustrated that there is a voltage drop from the center of the RF electrode to the periphery.

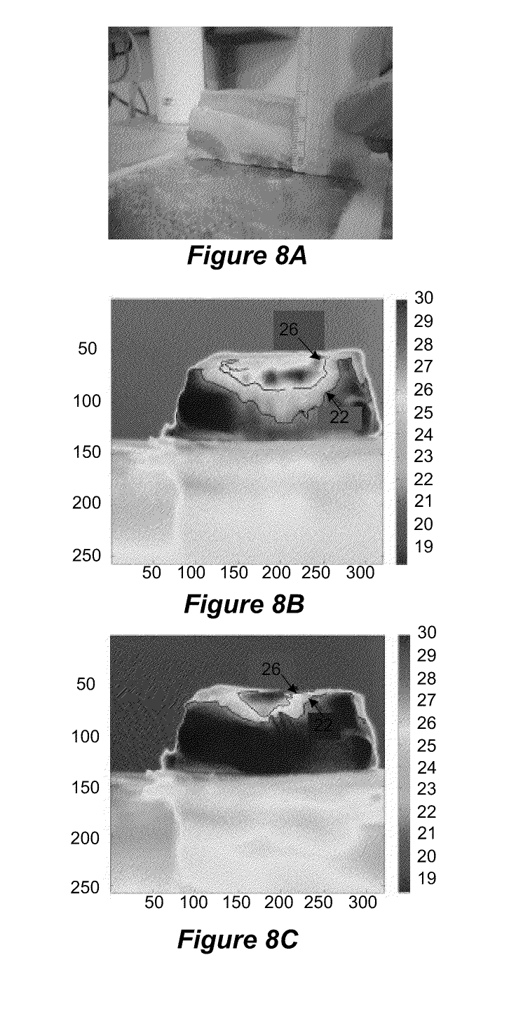

The corresponding normalized depth profiles of the electric field, E_{F,f}, and power absorption Q_{F,f}, within the subcutaneous fat layer are shown in FIG. 6. For simplicity, FIG. 6 shows the subcutaneous fat layer only. In other words, the coordinate origin is shifted to the top of the fat layer.

FIG. 6A illustrates the normalized internal electric field, E_{F,f}(0, z). FIG. 6B illustrates the power absorption, Q_{F,f}(0, z), within the subcutaneous fat layer along the center line of the RF electrode at five operational frequencies: 500 kHz, 1, 2, 3 and 4 MHz. Note that small variations in the internal electric field result in significant changes in power absorption, as shown by Equation (1). These are numerical curves obtained by mathematical modeling using the averaged experimental curves in FIG. 5 as boundary conditions. It is shown that increasing the frequencies leads to a higher internal electric field and higher power absorption in greater depths.

Changing the electric potential, V_{F,s}(x, 0), across the RF electrode from a uniform surface distribution, ΔV_{0.5,s}=V_{0.5,s} (0, 0)−V_{0.5,s}(L, 0)≈0.1, to a decreasing surface distribution from the center of the electrode, ΔV_{4,s}s≈0.35, changes the depth and lateral distribution of E_{F,f}(x, z) and, hence, the spatial distribution of Q_{F,f}(x, z), (see Equation (1) above). For a uniform surface distribution ΔV_{0.5,s}≈0.1, modeling shows that the difference between the electric field at the top and the bottom of the fat layer is ΔE_{0.5,f}=E_{0.5,f}(0, z1)−E_{0.5,f}(0, z2)≈0.22. The correspondent difference in power absorption is ΔQ_{0.5,f}≈0.4. For a decreasing surface distribution ΔV_{4,s}≈0.35, modeling shows that ΔE_{4,f}≈0.52 and ΔQ_{4,f}≈0.78. It follows that varying the operational frequency of the RF electrode results in significant changes in the depth distribution of fat heating, (FIGS. 5 and 6). Furthermore, the E_{F,f} and Q_{F,f} depth profiles are not linear and as the operational frequency increases the strength of the electric field also does (FIGS. 5 and 6). Thus, the heating intensity is higher at the upper fat layer.

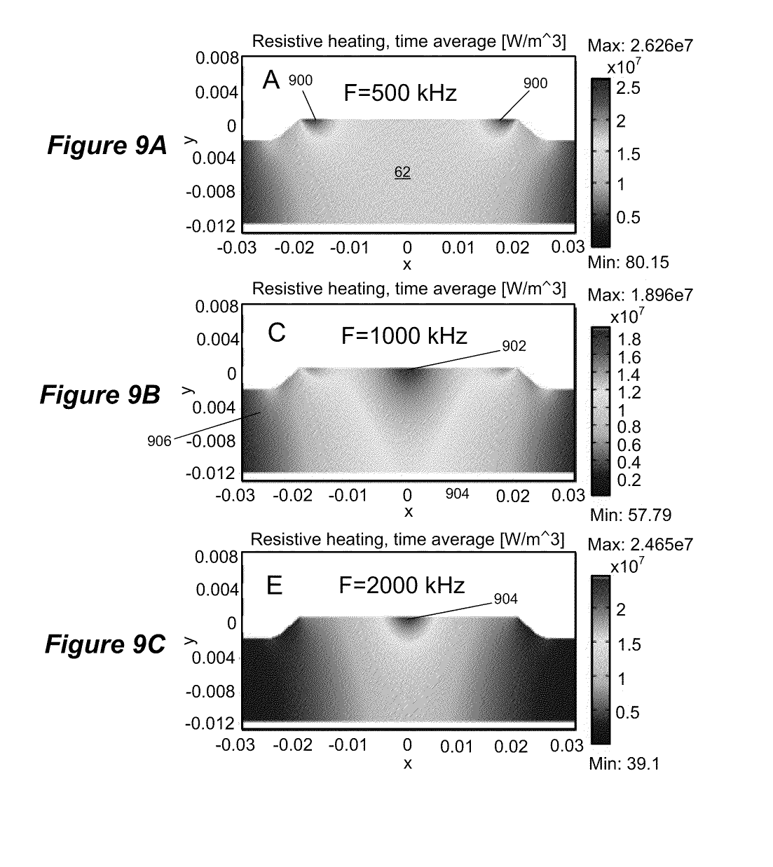

The electric field and power absorption relate to the heating of the tissue. Dynamics of the local tissue temperatures, T, beneath the center and edge of the RF electrode during exposure of ex vivo porcine tissue to 500 kHz and 4 MHz six-second RF pulses are shown in FIG. 7. FIG. 7A shows the local tissue temperature at the center of the RF electrode. A higher frequency leads to more heating than a lower frequency. Further, the center of the electrode leads to greater heating than the periphery of the electrode. FIG. 7B shows the local tissue temperature at the edge of the RF electrode. Temperature measurements were taken by microprobes placed 2 mm deep from the skin surface. Bars denote standard deviations of the mean (n=3) in one tissue sample.

Experimental temperature measurements showed that the difference between the power absorption at the center and periphery of the fat layer beneath the RF electrode increases as the operational frequency increases. The temperature difference between center and periphery at the end of the exposure is approximately 2° C. at 500 kHz and 7° C. at 4 MHz (FIGS. 7A and 7B). The rates of change are 2.2 and 1.6° C./s at the center and periphery, respectively, for 4 MHz. Therefore as the operational frequency increases the strength of the electric field and, thus, the heating intensity is higher at the center of the fat layer beneath the RF electrode.

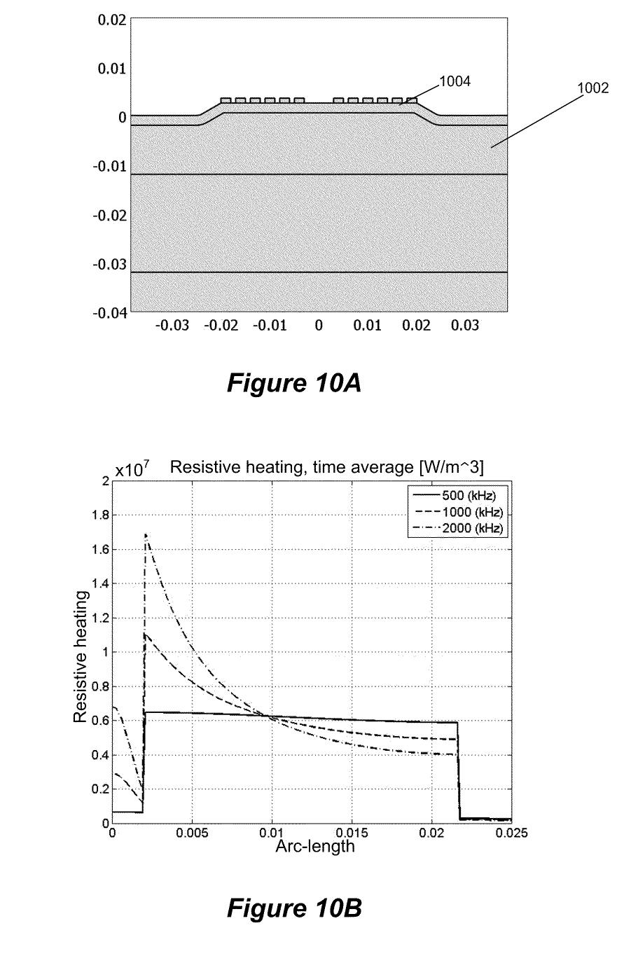

Cross-sectional radiometric temperature maps at the end of the RF exposures to the 500 kHz and 4 MHz pulses are shown in FIG. 8. FIG. 8A shows the experimental porcine tissue portion. FIG. 8B shows the cross-sectional experimental radiometric temperature map at the end of the 500 Hz RF exposure. FIG. 8C shows the cross-sectional experimental radiometric temperature map at the end of the 4 MHz RF exposure. Solid lines denote 22 and 26° C. isotherms. Units of temperature map and color side bar are a.u. and ° C., respectively. As illustrated in FIG. 8A, the lower frequency heats to a greater depth in a more uniform manner. FIG. 8B, the higher frequency, has greater heating near the surface.

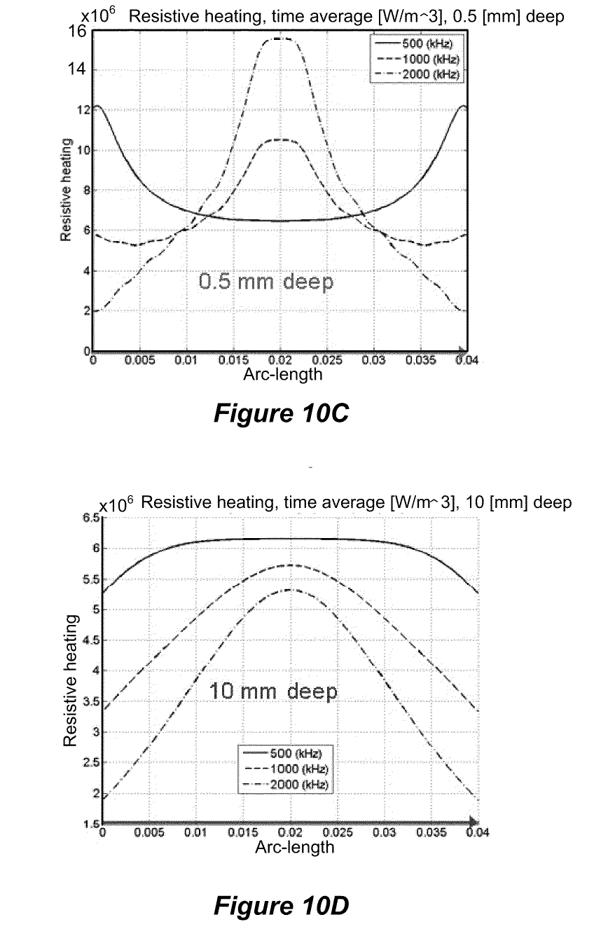

FIGS. 9A, 9B and 9C represent modeling of the effects of resistive type heating of tissue with different modulation frequencies. FIG. 9A shows the effects at 500 kHz. In FIG. 9A, the two hottest regions 900 are small and located near the outer edges of the where the electrode would couple into the tissue. A relatively large central region 62 is subjected to an even, mid-level heating. Region 62 is large in both width and depth. The coolest regions 64 are relatively small. FIG. 9B shows the effects at 1000 kHz (1 MHz). Here, there is a large, hot region 902 near the center that projects down into the tissue. The cooler regions 906 are increased somewhat over FIG. 9A. At 2000 kHz (2 MHz) shown in FIG. 9C, the hottest region 904 is located at the center and is very shallow. Much of the rest of the tissue is much cooler.

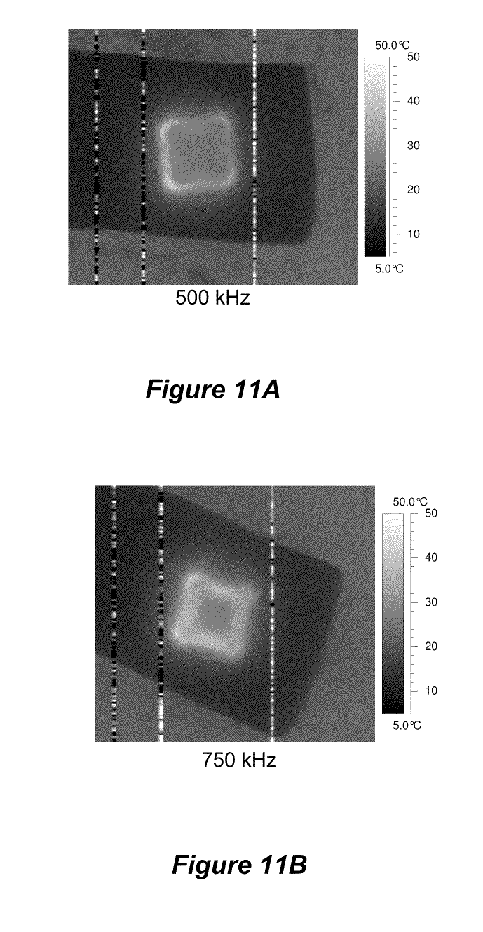

FIG. 10 represents the information of FIG. 9 in a different form. FIG. 10A shows tissue 1002 under an electrode 1004. FIG. 10B shows the level of resistive heating as a function of depth beneath the tissue for different frequencies with a cooled surface. As can be seen, resistive heating at 500 kHz is relatively flat over a large depth range. In contrast, as the frequency is increased, shallower depths are heated to a higher temperature while deeper depths are heated to a lower temperature (compared to 500 kHz). FIG. 10C shows a heating profile in a plane taken at a depth of 0.5 mm below the surface. It can be seen that at this shallow depth, the largest response occurs at 2000 kHz. In contrast, FIG. 10D shows the heating profile at a depth of 10 mm (twenty times the depth of FIG. 10C). Here, there is a generally uniform, maximum heating at 500 kHz while the heating caused by the 2000 kHz frequency is less and more confined to the center.

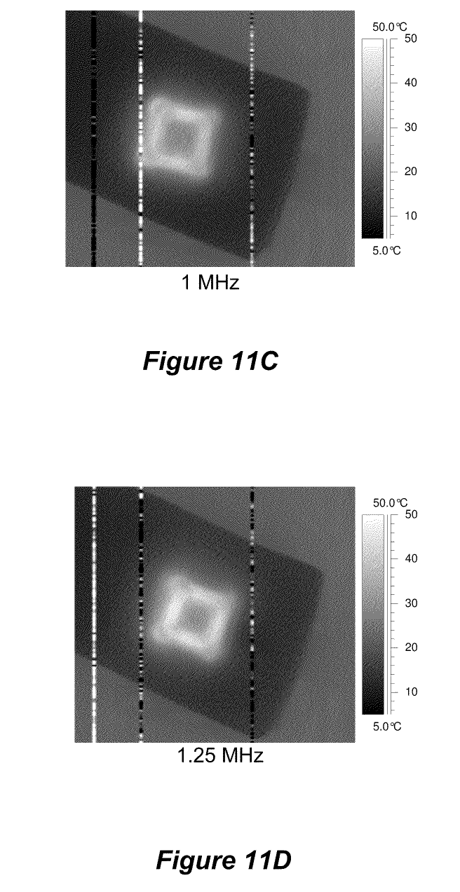

This effect has been explored experimentally. The photographs of FIG. 11 were taken with a thermal imaging camera of ex vivo porcine tissue after treatment at various frequencies with a handpiece similar to the one illustrated in FIGS. 2 and 3. FIG. 11A illustrates the response at 500 kHZ, FIG. 11B is the response at 750 kHz, FIG. 11C is the response a 1 MHz and FIG. 11D is the response at 1.25 MHz. The effect can best be seen comparing FIGS. 11A and 6D. In FIG. 11A, the heating is relatively uniform over the entire region. In contrast, the heating in FIG. 11D is much higher and more localized near the center of the electrode.

Based on these initially experiments and using a handpiece of the type shown in FIGS. 2 and 3, it is estimated that when treating a fat layer about 4 cm thick, one should use a relatively low RF frequency, in the range of 500 kHz. In contrast, for a fat layer on the order of about 1 cm, an RF frequency in excess of 1 MHz and possibly as high at 5 MHz can be used.

Furthermore, heating in the fat is greater than in skin and muscle if the RF electrode produces an electric field perpendicular to the skin-fat and fat-muscle interface. Using equation (1), we can derive the power absorbed per unit volume in fat and skin is, respectively:

$$\begin{matrix}{Q}_{F , i} = \frac{1}{2} {σ}_{F , s} {E}_{F , s}^{2} , & ( 4 ) \\ {Q}_{F , f} = \frac{1}{2 } {σ}_{F , f} {E}_{F , f}^{2} , & ( 5 )\end{matrix}$$

If the electric field is perpendicular to the skin-fat interface, then the boundary condition at this interface is:

ε_{F,s}E_{F,s}=ε_{F,f}E_{F,f}, (6)

where ε_{F,s} and ε_{F,f} are the relative complex permittivities of skin and fat, respectively. Combining Equations 4, 5 and 6 results in the following analytical expression for the fat-skin ratio of power absorption:

$$\frac{{Q}_{F , f}}{{Q}_{F , s}} = \frac{{σ}_{F , f}}{{σ}_{F , f}} \frac{{\vert {ɛ}_{F , s} \vert}^{2}}{{\vert {ɛ}_{F , f} \vert}^{2}} . \qquad ( 7 )$$

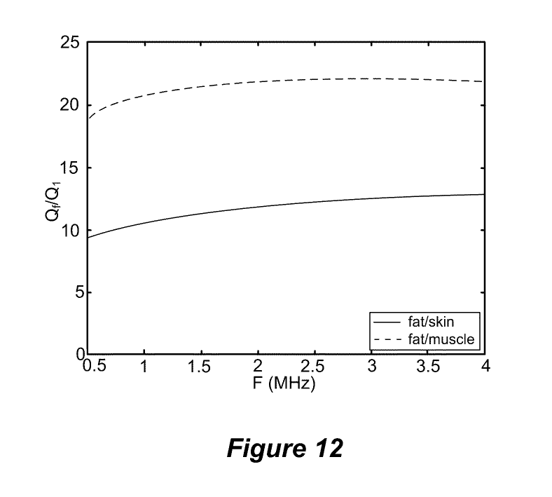

Similarly, expressions can be derived for the fat and muscle. Ratios of power absorption for the frequency range 500 kHz≦F≦4 MHz are shown in FIG. 12. The ratios of power absorption are shown as a function of frequency: Q_{f}/Qₛ (fat/skin) and Q_{f}/Qₘ (fat/muscle). Heating in fat is greater than in skin and muscle when E is perpendicular to the skin-fat and fat-muscle interface. These are analytical curves obtained with Equation (7) and the electric properties of tissue at different frequencies.

Note the selectivity in heating fat within this operational frequency range, Q_{F,f}/Q_{F,s}>9 and Q_{F,f}/Q_{F,m}>18. Even though the fat (σ_{f}=0.025-0.026) has less attenuation or dissipation of the electric field (lossy) than the skin (σₛ=0.18-0.31) and muscle (σₘ=0.45-0.58), it heats more because the internal electric field is stronger than those in skin and muscle. It follows that the epidermis, dermis and muscle are intrinsically protected from undesired power absorption. However, as the direction of the electric field deviates from perpendicularity this selectivity in heating is reduced. As mentioned above, surface cooling might be required to protect dermis and epidermis since more power is absorbed within these layers. Muscle would continue to be protected because of the attenuation in power absorption.

Within the frequency range considered herein there is power absorption throughout the entire fat layer. However, at relatively high frequencies power is transferred rapidly near the skin-fat interface, attenuating the energy deposition as power is taken out of it and creating smaller but more intense heating zones. At relatively low frequencies energy deposition is more uniform over a greater volume, resulting in uniform bulk heating of greater tissue volumes. Therefore high frequencies are appropriate for treatment of smaller areas while low frequencies are appropriate for larger areas.

In summary, having the ability to couple energy into tissue across the entire surface of the electrode and to control the surface distribution of the applied energy provide a unique treatment modality for which the subcutaneous fat is selectively heated and the size of the heated volume is controllable. Our preliminary results show that it is feasible to adjust the operational frequency in order to match anatomical structure from patient to patient or within the same patient. For example, body areas with thin fat layers would be treated using high frequencies while low frequencies would be used with thicker fat layers. FIG. 5 illustrates the difference in the extent of the subcutaneous fat heating volumes using high and low frequencies in a tissue model with 19 mm fat layer thickness. At 500 kHz, there is heating across the 19 mm layer. At 4 MHz, heating is confined to the first 7 mm of fat. Representative temperatures of the skin, fat and muscle along the centerline of the electrode are 25.2, 28.2 and 21° C., respectively, at 500 kHz and 27.5, 29.8 and 20, respectively, at 4 MHz. Hence similar temperatures can be reached with different frequencies although the spatial distribution is different.

In the most basic approach, the user, such as a physician, will determine the thickness of the fat tissue. This might be done through a visual observation, by pinching the skin or by using calipers. The user may also take a body measurement related to body weight. The body measurement can indicate body fat content. An example of a body measurement is body mass index (BMI), which is a statistical measurement based on a person's height and weight. Once the physician determines the thickness of the fat layer, or the body measurement indicating a measure of fat, he would select the appropriate frequency from the user input section 112 of the console 102. In other embodiments, the physician would input the thickness of the fat layer in user input section 112. The controller 114 would automatically select the appropriate frequency based on the thickness entered by the physician, or any other user. It is also possible that the console can be programmed so that the physician merely has to enter the thickness of the fat layer and the console will compute the best frequency to use under the circumstances. In either case, the RF frequency applied to the tissue is selected to best match the tissue thickness to produce an effective result without damaging any underlying tissue.

It is also envisioned that the system can be provided with a measurement system for determining the thickness of the fat layer. If such a measurement was made, the system could automatically select the best frequency for treatment.

One approach for measuring the depth of fat is an ultrasound technique. One or more transducers could be provided in the bottom of the handpiece for generating an ultrasound pulse directed into the tissue. The same or different transducer could be used to detect the reflected pulse. The pulse would typically be reflected by bone. One could measure the time of flight of the pulse to determine layer thickness. Alternatively, one could measure the amplitude of the pulse as a thinner fat layer will return a higher amplitude pulse than a thicker fat layer. In either case, the measurement would be used to select the best RF frequency.

Another approach for measuring the thickness of an underlying fat layer using the handpiece relies on the thermoelectric cooler (TEC) 52. More specifically, a TEC uses the Peltier effect to draw heat away from the skin surface and into the heat sink 56. The heat sink can be either air or water cooled.

A TEC can also work in reverse in that any heat flux passing though the module when it is turned off will generate a proportional voltage across its terminals. This reverse process is known as the Seebeck Effect. This Seebeck effect can be used to determine the heating of the tissue. If the amount of energy applied is known, and the amount of heat in the tissue is known, the processor may be able to determine the thickness of the underlying fat layer. For example, if the tissue is thinner, it will become hotter for a given amount of input energy. The Seebeck effect can also be used to determine a treatment end point. More specifically, when the heat content of the tissue reaches a certain level, the system can be programmed to turn off.

At the start of a procedure, the TEC is typically used to cool the surface of the tissue to a predetermined temperature, such as five degrees centigrade. During RF treatment, the thermistors 48 monitor the tissue temperature. The operation of the TEC is controlled in response to the temperature measurements in an effort to maintain the surface temperature as close as possible to the desired temperature. More specifically, if the temperature begins to rise, the power to the TEC can be increased. If the cooling provided by the TEC is insufficient to maintain the desired temperature, the power of the RF energy can be lowered.

In a preferred embodiment, the delivery of treatment energy is briefly and periodically interrupted, for example, every few seconds. During this time, the return heat flux is measured. In a first step, the drive current to the TEC is turned off while the RF energy is continued to be supplied to the electrode for a brief period allowing both sides of the TEC to reach approximately the same temperature. At this point, the delivery of RF energy is halted.

The voltage waveform across the TEC leads is then measured for a time comparable to the fat-to-surface thermal time constant. Thereafter, the drive current to the TEC is restarted to resume cooling of the treatment area followed by a resumption of the delivery of RF energy. The TEC waveform data can then be analyzed to derive information about fat layer thickness and/or whether tissue has been sufficiently heated so that treatment can be stopped.

The voltage waveform generated when the TEC drive voltage has been turned off will be opposite to the normal drive voltage when the tissue is warmer than the heat sink backing the TEC. Conversely, if the tissue is cooler than the heat sink temperature, the generated voltage will be the same polarity as the TEC drive voltage. In the discussion below, if the voltage is opposite the drive voltage it is referred to as positive. Therefore, a positive voltage indicates a net heat flow from the tissue towards the heat sink. Either polarity convention can be used in actual practice.

Based on the measurement, a number of conclusions can be drawn from the TEC waveform data. More specifically, a waveform with negative slope indicates that both shallow and deep tissue layers are cooler than the heat sink. A waveform with an initial positive slope followed by a long negative slope indicates shallow temperatures higher than the heat sink, with deep temperatures being cooler. A waveform with positive slope indicates that both shallow and deep tissue layers are warmer than the heat sink. The steepness of the slope indicates the total temperature differential between the tissue mass and the heat sink. Relative comparisons of the first-order and higher-order components of the slopes would indicate the degree of deep heating, independent of gain and offset calibrations.

It should be noted that in some cases, it may be desirable to treat the site with more than one RF frequency. As noted above, the subject system is designed to be able to select among a wide range of treatment frequencies. As also noted above, changing the frequency changes the heating pattern. Accordingly, if it were decided, based on the thickness of the tissue, that a treatment frequency of one megahertz was desired, it might be beneficial to vary the treatment frequency about a one megahertz center frequency to smooth out the tissue heating. This variation could be done in steps or by sweeping the frequency. The amount of the excursion from the center frequency could be as small is 500 hertz or as large as 500 kilohertz. Regardless of the approach used to sweeping the frequency, it is expected that the average or center frequency selected should correlate to the thickness of the fat layer.

It should also be noted that another variable parameter is power delivered to the electrode. The higher the power, the higher the temperatures that will be generated. Changing power can also somewhat influence of heating. Optimum power settings can be determined through experimentation.

While the subject invention has been described with reference to a preferred embodiment, various changes and modifications could be made therein, by one skilled in the art, without varying from the scope and spirit of the subject invention as defined by the appended claims.

What is claimed is:

1. A method of treating a layer of subcutaneous fat using radio frequency (RF) heating, the method comprising:
determining a thickness of a layer of fat of a portion of tissue to be treated;
selecting an RF frequency based on the determined thickness; and
applying the RF heating to the portion of tissue by applying RF energy at the selected RF frequency.

2. The method of claim 1, wherein the selected RF frequency is between 100 kilohertz and 10 megahertz.

3. The method of claim 2, wherein the selected RF frequency is between 500 kilohertz and 5 megahertz.

4. The method of claim 1, further comprising:
cooling the portion of tissue to a predetermined temperature before applying the RF heating.

5. The method of claim 1, further comprising:
cooling the portion of tissue to a predetermined temperature during the step of applying the RF heating.

6. The method of claim 1, wherein the step of applying the RF heating includes applying the RF energy in cycles.

7. The method of claim 1, wherein selecting the RF frequency based on the determined thickness of the layer of fat and a metric of damage to the portion of tissue.

8. The method of claim 1, wherein determining the thickness of the layer of fat is based on an ultrasound technique.

9. The method of claim 1, wherein determining the thickness of the layer of fat is based on the Peltier effect.

10. The method of claim 1, wherein the power of the RF energy is modified during the applying of the RF heating based on temperature measurements of the portion of tissue during the applying.

11. A system for treating a layer of subcutaneous fat using radio frequency (RF) heating, the system comprising:
a controller configured to generate an RF signal at a selected frequency, via an electrode, for RF heating of a portion of tissue, wherein the selected frequency is selected based on a determined thickness for a layer of fat included in the portion of tissue;
an electrode, connected to the controller, configured to apply the RF heating to the portion of tissue by applying the RF signal generated by the controller at the selected frequency, wherein the electrode is included in a handpiece.

12. The system of claim 11, further comprising:
means for determining the thickness of the layer of fat, and for generating a thickness signal in response thereto, the thickness signal being supplied to the controller for selecting the frequency of the RF signal based on the supplied thickness signal.

13. The system of claim 11, wherein the handpiece comprises a cooling apparatus configured to cool the portion of tissue.

14. The system of claim 13, wherein the cooling apparatus is further configured to determine the thickness of the layer of fat.

15. The system of claim 11, wherein the handpiece comprises thermistors for measuring a temperature of the portion of tissue.

16. The system of claim 11, wherein the handpiece comprises a suction apparatus for applying suction to the portion of tissue so that the handpiece securely contacts the portion of tissue.

17. The system of claim 11, wherein the controller further comprises a user interface configured for inputting, by a user, the thickness of the layer of fat.

18. A method of treating a layer of subcutaneous fat using radio frequency (RF) heating, the method comprising:
determining a thickness of a layer of fat below the surface of the skin;
selecting an RF frequency based on the determined thickness;
generating an RF signal at the selected frequency;
supplying the RF signal to an electrode which is placed into contact with the skin, whereby the electrode generates an RF energy field for heating the tissue and causing the fat to be reduced.

19. The method of claim 18, wherein the step of determining the thickness of the layer of fat is receiving a user input indicating the thickness of the layer of fat.
